(19) United States
(12) Patent Application Publication
(10) Pub. No.: US 2003/0180762 A1
(43) Pub. Date: Sep. 25, 2003

(54) Mild enrichment of foetal cells from peripheral blood and use thereof

(76) Inventors: Wolfgang Tuma, Gelsenkirchen (DE); Gerd Bunger, Gelsenkirchen (DE); Michael Pitone, Gelsenkirchen (DE); Heinrich Burrichter, Gelsenkirchen (DE)

Correspondence Address: LEYDIG VOIT & MAYER, LTD, 700 THIRTEENTH ST. NW, SUITE 300, WASHINGTON, DC, 20005-3960, US

(21) Appl. No.: 10/333,245

(22) PCT Filed: Jul. 17, 2001
(86) PCT No.: PCT/DE01/02723

(30) Foreign Application Priority Data
Jul. 20, 2000 (DE) 100 35 433.5

(51) Int. Cl. C12Q 1/68; G01N 33/567; C12N 5/08

(52) U.S. Cl. 435/6; 435/7.21; 435/372

(57) ABSTRACT

The invention relates to a procedure for high enrichment of fetal cells as a product, whereby after a withdrawal of maternal blood, the blood fraction and centrifugation, an antibody cocktail with Anti-w, Anti-r antibodies, which specifically bond to transferring receptor molecules as antigens of the surfaces of nucleated stem cells of red blood corpuscles, and Anti-i antibodies, which specifically bond to Antigen-i as antigens of the surfaces of nucleated stem cells of red blood corpuscles, and/or Anti i plus antibodies, which specifically bond to intracellular structures of fetal stem cells and/or the intracellular molecules of fetal stem cells and/or membrane fragments of fetal stem cells is added and fetal cells with 10⁵ to 10⁷ enrichment are separated from the cell preparation.

The invention relates to a procedure for enrichment of fetal cells as a product of, preferably peripheral, maternal blood, whereby in Step a) after a withdrawal of maternal blood in the presence of a solution containing one or more anticoagulants for provision of a blood fraction, the blood is diluted with an isotonic solution as well as in Step b) the blood fraction, for the purpose of enrichment of a cell fraction with nucleated fetal cells, is centrifuged, the use of the products for the treatment of diseases and for determination of gene and/or genome analyses.

In the state of the art procedures are known for isolating fetal cells. These are then analyzed for genome defects, hereditary diseases and the like. The isolation of fetal cells from the peripheral blood of the mother is desirable to help prevent the undesirable serious disadvantages of conventional procedures for the earliest possible detection of existing diseases or developmental anomalies, since the conventional methods based on the foundation of amniocentesis or chorionic villi biopsy do not rule out the risk of miscarriage and infections.

In addition, it is desirable to obtain fetal cells as gently as possible without altering their surface, e.g., without altering the primary structures, secondary structures and further structures of the compounds arranged on the surface, in the cell membrane, whose inalterability can serve as the measure for the gentle processing of the cells.

In contrast to the state of the art, the isolated fetal cells and their genome can be used for analysis of hereditary defects such as single-point mutations, chromosome anomalies, chromosome aberrations without the necessity of removing cells from the amniotic fluid or the uterine organ so that the risks of miscarriage can be ruled out.

The ceramide compounds are surface molecules of stem cells' red blood cells. It is assumed that during postnatal development, the polylactose aminopolysaccharides branch out. This maturation cycle of the carbohydrate chains, which also takes place in the preliminary stages of the erythrocytes in the bone marrow of adults, results in lessening of the Lacto-N-nor-hexaosyl-Ceramide compound as i-Antigen (also called Antigen-i) and results in the augmentation of the Lacto-N-iso-octaosyl-Ceramide compound as Antigen-I, also called I-Antigen. The Antigen-I is therefore only poorly developed, if at all, in births. Not until about 18 months does it reach the quantity and concentration that it has in adults. The i-Antigen is completely developed in newborns. It is consequently lost in the first 18 months after birth and is replaced by Antigen-I.

From this time on the need exists to provide procedures which make possible a high enrichment of fetal cells eliminating maternal blood cells and a gentle enrichment of the same. The DNA of the highly enriched fetal cells can give information about the primary structure up to the quaternary structures of the gene sequences, without the results of the analysis being influenced or even changed by the genome of the maternal blood cells. In addition, there is also the desire to provide products and cells from fetuses quickly and at any time without immediate intervention into the same, which could be used for treatment of diseases, such as hereditary diseases and their early detection and therapy.

For example, in U.S. Pat. No. 5,437,987 a procedure is described in which fetal red blood cells from maternal blood are enriched and complexes made of polyclonal antibodies and fetal cells are separated after addition of the antibody Anti-i; in this case the polyclonal antibodies bond to the surface antigens i of the fetal cells.

As antibodies, such are used whose antigen binding region specifically bonds with the Gal β-4-GlcNAc β-1-3-Gal β-1-4-GlcNAc β-1-3-Gal β-1-4-Glc-chain of the antigen as antigenic determinants. This antibody directed toward the linear carbohydrate chain requires at least two repeating N-Acetyl-Lactosamine-units for bonding. The simplest i-active structure is accordingly Lacto-N-nor-hexaosyl-Ceramide. The antibody which is able to bond itself to Lacto-N-nor-hexaosyl-Ceramide or derivatives of the same as an antigen is called Anti-i as defined by the invention.

In the conventional procedure magnet-activated cell sorting procedures are used for the purpose of separating the antibody-antigen complex, however, they are only able to enrich the fetal cells to an insufficient extent, since the fetal cells are present in an infinitesimal ratio and extent in the antibody-antigen complex.

The sought after cells are extremely rare in an excess of maternal cells, so that the fetal cells are enriched only in a slight degree, but not to purity. Due to the fact that the fetus-specific properties of the antibodies available up to now are lacking, appreciable so-called cellular impurities with maternal blood cells cannot be prevented. Moreover, the isolation of cells using magnet-activated cell sorting procedures is not possible.

Therefore the task is also to prepare a gentle purification procedure or an enrichment procedure which makes possible a high enrichment degree of fetal cells. These fetal cells can be used as products in a diagnostic procedure.

The task of the invention is solved by the main claim and the independent claim. The sub-claims relate to preferred embodiments and further developments of the invention.

The invention relates to a procedure for high enrichment of fetal cells as a product of, preferably peripheral, maternal blood, whereby in Step a) after a withdrawal of maternal blood in the presence of a solution containing anticoagulants for preparation of a blood fraction, the blood is diluted with an isotonic solution as well as in Step b) the blood fraction, for the purpose of enrichment of a cell fraction with nucleated fetal cells, is centrifuged, which is characterized by the fact that in Step c) an antibody cocktail made of antibodies of the polyclonal and/or monoclonal type, preferably at RT for 15 to 30 min, even more preferably 10 to 20 min long which contains Anti-w antibodies, which specifically bond to antigens of the intracellular structures of white blood corpuscles and/or the intracellular molecules of white blood corpuscles and/or surfaces of white blood corpuscles, and Anti-r antibodies, which specifically bond to antigens of the intercellular structures of nucleated stem cells of red blood corpuscles and/or the intracellular molecules of nucleated stem cells of red blood corpuscles and/or the surfaces of nucleated stem cells of red blood corpuscles, is added to the cell fraction as well as adding Anti-i antibodies of the polyclonal and/or monoclonal type, which specifically bond to Antigen-i as antigens of the surfaces of nucleated stem cells of red blood corpuscles, and/or Anti i plus antibodies of the polyclonal and/or monoclonal type, which specifically bond to intracellular structures of fetal stem cells and/or the intracellular molecules of fetal stem cells and/or membrane fragments of fetal stem cells, under formation of a cell preparation, which contains antibody-complexes that comprise Anti-r and Anti-i antibodies coupled to fetal cells, and if necessary Anti-i plus antibodies, and Step d) is separated from the cell-preparation fetal cells with Anti-r antibodies and/or Anti-i antibodies coupled to the same, preferably and/or Anti-i plus antibodies, on the basis of their diffuse light and fluorescent properties, the properties of surfaces and of cell interiors as well as the size distribution of the fetal cells with 10⁵ to 10⁷ enrichment of the fetal cells, preferably until their cellular isolation.

One object of the invention has to do with a use of the cell fraction as a product which can be manufactured according to the invention's procedure, for treatment of hereditary diseases, preferably in the fetal period, for use in diagnostic procedures, for example through genome research.

A further object of the invention comprises fetal cells of venous blood, which are producible through in Step a) dilution of maternal blood provided after withdrawal in the presence of an isotonic solution containing anticoagulants for provision of a blood fraction as well as Step b) centrifugation of the blood fraction for the purpose of enrichment of a cell fraction with nucleated fetal cells, Step c) incubation of the cell fraction with an antibody cocktail made of antibodies of the polyclonal and/or monoclonal type at RT for 15 to 30 min, preferably 10 to 20 min, long, which contains Anti-w antibodies, which specifically bond to antigens of intracellular structures of white blood corpuscles and/or intracellular molecules of white blood corpuscles and/or surfaces of white blood corpuscles, and Anti-r antibodies, which specifically bond to antigens of intracellular structures of nucleated stem cells of red blood corpuscles and/or the intracellular molecules of nucleated stem cells of red blood corpuscles and/or the surfaces of nucleated stem cells of red blood corpuscles, and with Anti-i antibodies of the polyclonal and/or monoclonal type, which specifically bond to Antigen-i as antigens of surfaces of nucleated stem cells of red blood corpuscles, and/or Anti i plus antibodies of the polyclonal and/or monoclonal type, which specifically bond to intracellular structures of fetal stem cells and/or the intracellular molecules of fetal stem cells and/or membrane fragments of fetal stem cells, under formation of a cell preparation made of antibody complexes which comprise Anti-r and Anti-i and/or Anti-plus antibodies coupled to fetal cells, Step d) separation of fetal cells from the cell-preparation fetal cells with Anti-r antibodies and/or Anti-i antibodies coupled to the same, preferably and/or Anti-i plus antibodies, on the basis of their diffuse light and fluorescent properties, the properties of surfaces and of cell interiors as well as the size distribution of the fetal cells with 10⁵ to 10⁷ enrichment of the fetal cells, preferably until their cellular isolation.

As defined by the invention, by the intracellular structures of white blood corpuscles or of fetal stem cells, organelles such as mitochondria, endoplasmic reticulum and cell nuclei are understood.

As defined by the invention, by the intracellular molecules of white blood corpuscles or of fetal stem cells, cell-specific products or compounds, for example proteins such as enzymes, protein derivatives such as fetal hemoglobin are understood.

As defined by the invention, by antigens of the surfaces of nucleated stem cells of red blood corpuscles, transferrin receptor molecules are understood.

As defined by the invention, by adding or addition the adding of antibody cocktails and the Anti i and Anti i plus antibodies simultaneously or in succession is understood.

For example, first the antibody cocktail of the cell fraction can be added, then the Anti i antibodies and finally the Anti i plus antibodies can be added in succession.

It is also possible to add first the antibody cocktail of the cell fraction, then the Anti i plus antibodies and finally the Anti i antibodies can be added one after the other.

You can also add the antibody cocktail, the Anti i antibodies and the Anti i plus antibodies of the cell fraction at the same time.

As defined by the invention, by adding or addition the adding of antibody cocktails simultaneously or in succession it is also understood, for example, that in Step c) first the antibody cocktail is added as a mixture of Anti-w antibodies and Anti-r antibodies of the cell fraction or in Step c) the Anti-w antibodies of the cell fraction are added first, followed by the Anti-r antibodies, or in Step c) the Anti-r antibodies of the cell fraction are added first, followed by the Anti-w antibodies.

As defined by the invention, by incubation, the addition, either simultaneously or in succession, of antibody cocktail and Anti i and Anti i plus antibodies is understood, whereby in Step c) the antibody cocktail can be added first as a mixture of Anti-w and Anti-r antibodies of the cell fraction or in Step c) the Anti-w antibody of the cell fraction can be added first, followed by the Anti-r antibody or in Step c) the Anti-r antibody of the cell fraction is added first, followed by the Anti-w antibody in Step c) the additions of the antibody cocktail, the Anti-i antibody or the Anti-i-plus antibody or mixtures of the Anti-i antibody or the Anti-i-plus antibody can occur either simultaneously or one after the other. In the same way, mixtures can contain Anti-i antibodies of the polyclonal and/or monoclonal type and/or Anti i plus antibodies of the polyclonal and/or monoclonal type.

In the invention's procedure, a blood fraction is withdrawn from maternal, preferably peripheral, such as venous and/or arterial, blood and transferred to a container which has one or more conventional complexing agents as anticoagulants. Ethylenediaminetetraacetic acid compounds, heparinic acid or citric acid compounds and/or salts of the same and/or derivatives of the same can be used as complexing agents. Ethylenediaminetetraacetate compounds or their derivatives in concentrations familiar to the specialist serve as suitable anticoagulants without intermediate effects in the subsequent processing steps.

By fetal cells, which can be enriched and can be enrichable in accordance with the invention's procedure, fetal nucleated stem cells of erythrocytes are understood as defined by the invention. The fetal cells to be enriched have a nucleus and a transferrin receptor.

By transferrin receptors, as defined by the invention, those compounds are understood which serve the function of transporting ferric ions to the cell and which can no longer be found on the cell surfaces of the fully developed erythrocytes. Transferrin is a transporter of ferric ions to the cell.

The fully developed cells of the stem cells of the red blood corpuscles, namely the non-nucleated erythrocytes, have essentially no transferring receptors on their surface.

The blood fraction is subjected to a density gradient centrifugation step. In the density gradient procedure the nucleated fetal stem cells can essentially be enriched from the other cells out of the fraction on the basis of their variable density, however at the same time there are also erythrocytes in the cell fraction due to the so-called streaking occurring in all fractions.

Under the influence of gravity in accordance with their density, the nucleated cells concentrate themselves into a boundary layer, also known as a buffy-coat layer, between the upper fraction, the diluted blood plasma of the blood fraction and the lower fraction with a part out of sucrose as a cushion.

The sucrose polymer part can, for example, be a synthetic polymer made of sucrose with a molecular weight of 70,000 to 400,000 daltons, preferably 400,000 daltons, e.g. Ficoll, preferably colloidal, polyvinylpyrrolidone coated silica particles, such as Percoll, or the like. The sucrose polymer part is located in an isotonic solution of buffered physiological sodium chloride solution with pH values between 7.2 to 7.4, preferably 7.2.

Conventional buffers containing sodium chloride can be used as buffers, such as potassium phosphate and/or sodium phosphate buffers, NaH₂PO₄, Na₃HPO₄ with or without NaN₃.

For example, the cushion can be coated with a layer of isotonic sucrose-polymer solution with the blood fraction, if necessary after its preceding dilution with a volume ratio of 1:1 (Vol:Vol) with phosphate-buffered sodium chloride buffer solution; 1 part by volume blood fraction is given to 1 part by volume sucrose-polymer solution.

After a centrifugation of up to 60 min, preferably approximately 15 to 60 sec, even more preferably 45 sec, long with 800×g the red, but physically denser erythrocytes sediment in the sucrose cushion. The less dense nucleated fetal cells accumulate as a so-called buffy-coat layer at the boundary layer between the diluted blood fraction located above the cushion and the denser lower cushion. The buffy-coat layer, regularly visible as a white ring, is transferred to a container, and the cell fraction of the buffy-coat layer is washed with a phosphate-buffered sodium chloride buffer solution.

Then the washed cell fraction is added to an antibody incubation. The cell fraction can contain:

fetal nucleated stem cells of the red blood corpuscles, which have i-antigen and antigen-r and/or antigen-i-plus, some fetal non-nucleated red blood corpuscles, hence fully developed, which contain i-antigens and no transferrin receptors, some maternal nucleated stem cells of the red blood corpuscles, which have I-Antigen and the transferrin receptors, and some maternal red non-nucleated blood corpuscles, hence fully developed, which have I-Antigens and no transferrin receptors.

The nucleated cells of the cell fraction which are enriched on the basis of the density gradient procedure have as a rule a high number of maternal cells (1 to 4×10⁷ cells) in comparison to the 20 to 50 fetal cells in the cell fraction.

In the subsequent enrichment step, for example an antibody cocktail is added to various antibodies of the monoclonal type and/or polyclonal type in order to provide a cell preparation of antibody complexes. Thus also in Step c) the antibody cocktail can be added first as a mixture of Anti-w antibody and Anti-r antibody of the cell fraction or in Step c) first the Anti-w antibody of the cell fraction is added and then the Anti-r antibody or in Step c) first the Anti-r antibody of the cell fraction is added and then the Anti-w antibody.

In this way a procedure for high enrichment of fetal cells can be provided as the product of, preferably peripheral, maternal blood, whereby in Step a) after a withdrawal of maternal blood in the presence of a solution containing anticoagulants for preparation of a blood fraction, the blood is diluted with an isotonic solution as well as in Step b) the blood fraction, for the purpose of enrichment of a cell fraction with nucleated fetal cells, preferably by means of density gradients, is centrifuged, which is characterized by the fact that in Step c) an antibody cocktail made of antibodies of the polyclonal and/or monoclonal type, preferably in excess, at RT for 5 to 30 min, preferably 10 to 20 min, or 5 to 10 min long which contains Anti-w antibodies, which specifically bond to antigens of the surfaces of white blood corpuscles, Anti-r antibodies, which specifically bond to the transferrin receptor molecule as antigens of the surfaces of nucleated stem cells of red blood corpuscles, and/or Anti-i antibodies, which specifically bond to Antigen-i as antigens of the surfaces of nucleated stem cells of red blood corpuscles, and preferably Anti i plus antibodies, which specifically bond to membrane fragments of fetal stem cells, is added to the cell fraction under formation of a cell preparation, which contains antibody-complexes that comprise Anti-r and Anti-i antibodies coupled to fetal cells, and if necessary Anti-i plus antibodies, and Step d) is separated from the cell-preparation fetal cells with Anti-r antibodies and/or Anti-i antibodies coupled to the same, preferably and/or Anti-i plus antibodies, on the basis of their diffuse light and fluorescent properties, the properties of surfaces and of cell interiors and/or the size distribution of the fetal cells with 10⁵ to 10⁷ enrichment of the fetal cells, preferably until their cellular isolation.

In the same way, a use of the cell fraction can be provided as a product which can be manufactured according to the invention's procedure, for treatment of hereditary diseases, preferably in the fetal period, for use in diagnostic procedures, for example through genome research.

Likewise fetal cells of venous blood can be provided which are producible through in Step a) dilution of maternal blood provided after withdrawal in the presence of an isotonic solution containing anticoagulants for provision of a blood fraction as well as Step b) centrifugation of the blood fraction, preferably density gradient centrifugation, for the purpose of enrichment of a cell fraction with nucleated fetal cells, Step c) incubation of the cell fraction with an antibody cocktail made of antibodies of the polyclonal and/or monoclonal type at RT for 5 to 30 min, preferably 10 to 20 min, or 5 or 10 min long, which contains Anti-w antibodies, which specifically bond to antigens of the surfaces of white blood corpuscles, Anti-r antibodies, which specifically bond to transferrin receptor molecules as antigens of the surfaces of nucleated stem cells of red blood corpuscles, and Anti-i antibodies, which specifically bond to Antigen-i as antigens of the surfaces of nucleated stem cells of red blood corpuscles, and/or Anti i plus antibodies, which specifically bond to membrane fragments of fetal stem cells, under formation of a cell preparation made of antibody complexes which comprise Anti-r and Anti-i and/or Anti-plus antibodies coupled to fetal cells, Step d) separation of fetal cells from the cell-preparation fetal cells with Anti-r antibodies and/or Anti-i antibodies coupled to the same, preferably and/or Anti-i plus antibodies, on the basis of their diffuse light and fluorescent properties by density, the properties of surfaces and of cell interiors as well as the size distribution of the fetal cells with 10⁵ to 10⁷ enrichment of the fetal cells, preferably until their cellular isolation.

The antibody cocktail can, in an embodiment of the invention's procedure, comprise antibodies of the monoclonal type and/or polyclonal type: Anti-w antibodies against surface antigens, which are specific for white blood corpuscles, such as leukocytes, lymphocytes, and Anti-r antibodies against surface antigens, here against the transferrin receptors specific for red, still nucleated stem cells of the red blood corpuscles Anti-i antibodies against surface antigens, against the Antigen-i and/or Anti-i-plus antibodies against membrane fragments of fetal stem cells, which are specific for nucleated cells.

On the basis of the addition of the antibody cocktail, nucleated leukocytes are marked with the Anti-w antibodies, through the binding of the Anti-w antibodies to white blood corpuscles, such as leukocytes, lymphocytes, which are discarded.

In another enrichment step of the invention's procedure, for example, an antibody cocktail is added to various antibodies of the monoclonal and/or polyclonal type, to provide a cell preparation of antibody complexes.

The antibody cocktail of the invention's procedure can comprise antibodies of the monoclonal type and /or polyclonal type: Anti-w antibodies, which specifically bond to antigens of the intracellular structures of white blood corpuscles and/or the intracellular molecules of white blood corpuscles and/or surfaces of white blood corpuscles, such as leukocytes, lymphocytes, and Anti-r antibodies, which specifically bond to antigens of the intracellular structures of nucleated stem cells of red blood corpuscles and/or the intracellular molecules of nucleated stem cells of red blood corpuscles and/or the surfaces of nucleated stem cells of red blood corpuscles, for example which are specific against surface antigens, as against the transferrin receptors for red, still nucleated stem cells of the red blood corpuscles.

The polyclonal and/or monoclonal Anti-i antibodies against the Antigen-i and polyclonal and/or monoclonal Anti-i-plus antibodies, which specifically bond to the intracellular structures of fetal stem cells and/or the intracellular molecules of fetal stem cells and/or membrane fragments of fetal stem cells, which are specific for fetal nucleated cells, can be added to the antibody cocktail before the cocktail is added to the cell fraction, for example when its simultaneous incubation with the cell fraction is intended to occur.

In the same way, only the Anti-i antibodies can be added to the antibody cocktail, for example if the incubation with the cell fraction is to take place simultaneously. Likewise, only the Anti-i-plus antibodies can be added to the antibody cocktail, for example if the incubation with the cell fraction is to take place simultaneously.

In an incubation which is to occur in succession, the antibody cocktail can be added to the cell fraction first, then after the incubation of the cell fraction with the antibody cocktail, the Anti-i-plus or the Anti-i antibodies can be added to the cell fraction antibody cocktail or the antibody cocktail can be added to the cell fraction first, then after the incubation of the cell fraction with the antibody cocktail, the Anti-i- together with the Anti-i-plus antibodies can be added to the cell fraction antibody cocktail.

Furthermore, fetal, non-nucleated red blood corpuscles are marked as a result of the bonding of the Anti-i antibodies to the Antigen-i, which can be discarded.

Furthermore, the fetal, still nucleated stem cells of the red blood corpuscles that are to be enriched are marked, through the bonding of the Anti-r antibodies due to their bonding e.g., specifically bonding to antigens of the intracellular structures of nucleated stem cells of red blood corpuscles and/or antigens of the intracellular molecules of nucleated stem cells of red blood corpuscles and/or antigens of the surfaces of nucleated stem cells of red blood corpuscles, as to the transferrin receptors or their bonding to antigens of the surfaces that are specific for nucleated stem cells of red blood corpuscles, and through the bonding of the Anti-i antibodies to the Antigen-i and/or if necessary, in order to preferably increase the specific enrichment of the fetal cells even more appreciably, through the bonding of the Anti i plus antibodies to intracellular structures, which specifically bond for fetal stem cells and/or the intracellular molecules of fetal stem cells and/or membrane fragments of fetal stem cells.

Thus the fetal, still nucleated stem cell of a red blood corpuscle that is to be enriched can, for example, have both Anti-i and Anti-r antibodies on its surface. In the same way, the fetal, still nucleated stem cell of a red blood cell that is to be enriched can, for example have both Anti-i, Anti-r and Anti-i plus antibodies on its surface, for example to increase the specific cellular separation in the subsequent Step d).

The maternal matured erythrocytes cannot be marked by the Anti-w, Anti-r, Anti-i and/or Anti i plus antibodies due to a lack of transferrin receptors and Antigen-i and/or Antigen-i-plus or the Anti-w, Anti-r, Anti-i and/or Anti i plus antibodies, which are to be discarded, cannot specifically bond to them.

In a special embodiment of the invention's procedure or the preparation of fetal cells a further intermediate step prior to Processing Step d) can be carried out: this procedure of magnetic enrichment or depletion of cells uses commercially available antibodies, which e.g. partially fall under the described Anti-r and Anti-w antibodies; however, in this procedure the antibodies cannot contain the Anti-i or Anti-i-plus species. Through the additional use of a magnet-activated cell sorting procedure with antibodies of the type mentioned above, e.g. Anti-w as well as Anti-r, after a centrifugation the invention's procedure can be accelerated in the sorting. Also, the use of the magnet-activated cell sorting, in combination with the above-mentioned processing steps of the invention's procedure or with the preparation of the invention's fetal cells makes it possible to accelerate the purification of fetal cells.

In an additional processing step the cell preparation is subjected to the flow cytometer procedure. In this procedure different cells marked by means of antibodies can be differentiated to separate variable light scattering properties and/or different emitted fluorescent properties and separated from one another on the basis of the marked antibodies and the cells.

The cells marked correspondingly with antibodies are stimulated in the by-pass flow procedure as single cell suspension in a measuring chamber with laser light and the light is then characterized by means of transmitted light, diffuse light, and fluorescent detectors. Furthermore the liquid flow passes through deflector plates and the cells characterized as fetal with the help of the properties cited above receive an electric impulse, which deflects them laterally from the fluid. With this the fetal cells can be sorted until purity among other things as single cells in corresponding receptacles such as microtiter plates.

On the basis of the flow cytometer procedure fetal erythrocyte stem cells are gently enriched as fetal cells with the surface antigen i and/or surface antigen i plus in sufficient manner.

The antibodies of the polyclonal and/or monoclonal type can be induced through antigens isolated from cells, tissues or organs or through purely prepared antigens such as native antigens, and/or through chemically synthesized antigens.

The antibodies provided for the antibody cocktail step are for example those that are able to react as Anti-i specifically with Lacto-N-nor-hexaosyl-Ceramide compounds of the native type (isolable from cells) and/or of the synthetic type (chemically producible) or with derivatives of the same of the native type (isolable from cells) and/or of the synthetic type (chemically producible) and/or as Anti i plus specifically with intracellular structures of fetal stem cells and/or of intracellular molecules of fetal stem cells and/or membrane fragments of fetal stem cells of the red blood corpuscles.

The antibodies with specificity for Anti-i can belong to the IgM class.

As defined by the invention, an antibody is also understood as one that can react specifically with the Antigen i. Specific antibodies can prepare immunoglobulins, which are produced as the result of antigenic stimulation.

The polyclonal antibodies can be producible as vertebrates of any species, such as horses, sheep, mice and/or rabbits or the like. The monoclonal antibodies can be producible as e.g. B-Lymphocytes of vertebrates of any species, such as horses, sheep, mice and/or rabbits.

As defined by the invention, those immunoglobulins that are specifically directed only toward a single antigen determinant, such as Lacto-N-nor-hexaosyl-Ceramide compounds and/or derivatives of the same, which can be chemically synthesized or produced from cells native and pure, are also understood as monoclonal antibodies.

The Anti-w, Anti-r and Anti-i Anti-i plus monoclonal antibodies can be synthesized with conventional hybridoma techniques. In this process a single antibody-producing B-Lymphocyte is cloned in culture, so that monoclonal Anti-i, Anti-r or Anti-w or Anti-i plus antibodies can be obtained in large quantities. For example, the B-Lymphocytes of a mouse immunized against the Lacto-N-nor-hexaosyl-Ceramide compound as antigen are fused with cells of an infinitely divisible B-Lymphocyte tumor. From the resulting fusion products through the use of conventional selection media those hybrid cells are selected which, on the one hand e.g. produce the Anti-i antibody and on the other hand, have acquired the capability of dividing themselves infinitely in culture. Every single one of these hybridoma cells is the starting point of an individual cell clone, which grows permanently and in the process, produces a specific monoclonal antibody.

The Anti-w, Anti-r Anti-i and/or Anti-i plus monoclonal antibodies can be coupled simultaneously with fluorescein-isothiocyanate compounds (FITC), PE or the like for provision of fluorochrome antibodies. Even in a state of great dilution the antibodies, which can be coupled to the antigen, can be detected on the basis of the fluorescent marking and increase the yield of the cell sorting procedure.

Polyclonal antibodies that can be induced and isolated by methods known to specialists in vertebrates such as mammals, rats, rabbits, sheep, horses etc. can also be used as Anti-w, Anti-r. Anti-i and Anti-i plus antibodies. For example, the Anti-i antibody can be induced and isolated by methods known to specialists by adding a Lacto-N-nor-hexaosyl-Ceramide compound of the native and/or synthetic type and/or derivatives of the native and/or synthetic type in vertebrates, for example mammals such as mice, rats, rabbits, sheep, horses etc.

In the same way Anti-i plus polyclonal antibodies can be a mixture of various species of antibodies, whereby they can be induced and enriched or isolated in the conventional manner by the administration of intracellular structures of fetal stem cells and/or the intracellular molecules which are specific for fetal stem cells, and/or membrane fragments, which are specific for fetal stem cells, for example, the membrane fragments of fetal, preferably nucleated, cells, such as fetal stem cells of the red blood corpuscles, in vertebrates, e.g. mammals, such as mice, rats, rabbits, sheep, horses etc. The mixture can, for example, be one species as antibody, which is specific against Antigen-i or bonds with Antigen-i and contain one or more species which is specifically able to bond or react to a surface molecule or to surface molecules of the membrane fragments of fetal cells, such as fetal stem cells.

The antibody cocktail can contain fluorescein, phycoerythrine and/or peridinin chlorophyll protein (PerCP) marked antibodies (20 μl per antibody) of the monoclonal type, the polyclonal type or mixtures of the same with 0.1 to 10.00 μg, preferably −0.2 to 5.0, even more preferably 0.2 μg, protein/μl antibody cocktail, preferably in PBS.

The antibody cocktail can contain the following as antibodies for simultaneous incubation with the cell fraction μl Anti-w antibodies with 0.1 to 10.00 μg, preferably 0.2 to 5.0, even more preferably 0.2 μg, protein/μl antibody solution, preferably in PBS, which specifically bond to antigens of the surface of white blood corpuscles, 20 μl Anti-r antibodies with 0.1 to 10.00 μg, preferably 0.2 to 5.0, even more preferably 0.2 μg, protein/μl antibody solution, preferably in PBS, which specifically bond to antigens of the surfaces of the nucleated stem cells of red blood corpuscles, and 20 μl Anti-i antibodies with 0.1 to 10.00 μg, preferably 0.2 to 5.0, even more preferably 0.2 μg, protein/μl antibody solution, preferably in PBS, which specifically bond to the fetal Antigen-i on red stem cells.

The mixture ratio of antibody cocktail to cell preparation can amount to 0.1 to 100.0 μl, preferably 10.0 to 60.0 μl, preferably 20.0 to 40.0 μl even more preferably 20.0 μl, antibody cocktail to essentially 10⁵-10⁹, preferably 10⁷ nucleated cells in the cell preparation.

In a further preferred preparation the antibody cocktail can contain the following as antibodies for simultaneous incubation with the cell fraction: 20 μl Anti-w antibodies with 0.1 to 10.00 μg, preferably 0.2 to 5.0, even more preferably 0.2 μg, protein/μl antibody solution, preferably in PBS, which specifically bond to antigens of the surface of white blood corpuscles, 20 μl Anti-r antibodies with 0.1 to 10.00 μg, preferably 0.2 to 5.0, even more preferably 0.2 μg, protein/μl antibody solution, preferably in PBS, which specifically bond to antigens of the surfaces of the nucleated stem cells of red blood corpuscles, and 20 μl Anti-i antibodies with 0.1 to 10.00 μg, preferably 0.2 to 5.0, even more preferably 0.2 μg, protein/μl antibody solution, preferably in PBS, which specifically bond to the fetal Antigen-i on red stem cells, and/or 20 μl Anti-i plus antibodies with 0.1 to 10.00 μg, preferably 0.2 to 5.0, even more preferably 0.2 μg, protein/μl antibody solution, preferably in PBS, toward, membrane fragments of fetal red stem cells, preferably also containing Antigen-i on the membrane surface, which are specific for the fetal nucleated cells.

Likewise, the antibodies can also be added in succession to the cell fraction in the above specified concentrations and quantitative ratios, for example as additions in Step c) first the antibody cocktail as a mixture of Anti-w and Anti-r antibodies and then Anti-i and/or Anti-i plus or in Step c) first the antibody cocktail as a mixture of Anti-w and Anti-r antibodies and then Anti-i plus and/or Anti-i, or in Step c) first the Anti-w antibody and then the Anti-r antibody, then Anti-i and/or Anti-i plus or in Step c) first the Anti-w antibody and then the Anti-r antibody, then Anti-i plus and/or Anti-i, or in Step c) first the Anti-r antibody and then the Anti-w antibody, then Anti-i and/or Anti-i plus or in Step c) first the Anti-r antibody and then the Anti-w antibody, then Anti-i plus and/or Anti-i.

The Anti-i plus antibodies can react specifically with Antigen-i; Anti-i plus can also be a mixture of different species of antibodies, whereby e.g. the one antibody bonds specifically against Antigen-i and further antibodies bond specifically to surface molecules of the membrane fragments of fetal cells, such as fetal stem cells of the red blood corpuscles, or are able to bond with them. In this special embodiment of the invention's procedure the mixture ratio of antibody cocktail to cell preparation can also amount to 0.1 μl to 100.0 μl, preferably 10.0 to 60.0 μl, preferably 20.0 to 40.0 μl, even more preferably 20.0 μl, antibody cocktail 10⁵ to 10⁹, preferably 10⁷ nucleated cells in the cell preparation.

The Lacto-N-nor-hexaosyl-Ceramide compound is a naturally occurring substance and a surface antigen which can either be isolated from stem cells as a compound of the native type or can be chemically synthesized as a compound of the synthetic type and is an indicator for fetal cells, which cannot be detected in the blood of healthy mature human beings. Lacto-N-nor-hexaosyl-Ceramide compound is notated as a rule with the name i. This is a chain of sugar molecules, which is a linear, straight carbohydrate chain of repeating N-Acetyllactosamine units. The simplest i-active glucose phingolipid is Lacto-N-nor-hexaosyl-Ceramide.

It is also possible to use antibodies in the antibody cocktail which react specifically against Lacto-N-iso-octaosyl-Ceramide compounds and/or against their derivatives.

Since in healthy mature human beings the Antigen i is not detected on cells of the erythropoiesis, that is Lacto-N-nor-hexaosyl-Ceramide compound, this Antigen i is a suitable fetal marker for cells of the peripheral blood. All tests show that adults have no Antigen I on cells of the erythropoiesis, while Antigen i can be found as a surface compound in fetuses, newborn children and small children, as well as in the blood of pregnant women.

The antibodies contained in the antibody cocktail are monoclonal antibodies with uniform monospecifity. These monoclonal antibodies are synthesized in accordance with conventional steps familiar to specialists. Since the Antigen i can have limited immunogenic effect as a sugar molecule, antibodies against the Antigen i are created in an experimental animal in the following manner:

1. Against the native form of the human Lacto-N-nor-hexaosyl-Ceramide compound.

For this purpose pure cells are isolated from the blood of a newborn by means of a conventional high-performance cell sorter in time-consuming manner (e.g. Becton Dickinson, Vantage SE), which have i as the antigen Lacto-N-nor-hexaosyl-Ceramide compound on the surface of the cells. These cells are used both for the production of polyclonal antiserums, e.g. in rabbits, as well as for the production of monoclonal antibodies, for example in the mouse.

2. It is also possible to produce antibodies against the chemically synthesized form of the Antigen i Lacto-N-nor-hexaosyl-Ceramide compound in accordance with methods familiar to the specialist, whereby Antigen i is produced in pure form and can be used as described under 1.

The advantages of the invention's procedure in contrast to the conventional procedure are, among others,

The possibility of enriching fetal cells without impurities with maternal cells, the isolation of the fetal cells from the, preferably peripheral, maternal blood through simple puncture of a vein without the danger of infection of the abdominal cavity, the provision of cells provided in accordance with the invention's procedure for use in testing procedures of gene sequences, for treating diseases, for prenatal examination, for example for hereditary defects such as single-point mutations, chromosome anomalies, aneuploidies without the necessity of removing cells from the amniotic fluid or the uterine organ, the provision of cells for use in early detection examinations for the earliest possible detection of existing diseases or developmental disturbances, with no risk-attended procedures, e.g. amniocentesis, chordocentesis, or chorionic villi biopsy, as well as the reduction of the risks of miscarriage.

Practical Examples

Preferred embodiments and improvements of the invention's teaching are illustrated in conjunction with the illustrations of the preferred practical examples in schematic, greatly enlarged fashion:

Withdrawn venous maternal blood is diluted in an isotonic phosphate buffer solution containing anticoagulants pH 7.2 0.9% (w/v) NaCl, 150 mM NaH₂PO₄/Na₃HPO₄, preferably with NaN₃ for preservation, (PBS) at 25° Celsius to provide a blood fraction. The blood fraction is centrifuged at 800×g for 445 sec long for the purpose of enriching the cell fraction with nucleated fetal cells as a buffy-coat-layer at room temperature.

Then the whitish buffy-coat-layer as cell fraction (e.g. 1-4×10⁷ cells) is incubated with an antibody cocktail made of antibodies of the monoclonal type at room temperature for 20 min long to isolate the cell preparation.

The antibody cocktail contains fluorescein, phycoerythrine and/or peridinin chlorophyll protein (PerCP) marked antibodies (20 μl per antibody) of the monoclonal type with 0.2 μg, Protein/μl antibody cocktail in PBS.

The antibody cocktail contains the following in a practical example as antibodies for simultaneous incubation with the cell fraction:

20 μl Anti-w antibodies with 0.2 μg, protein/μl antibody solution, preferably in PBS, which specifically bond to antigens of the surface of white blood corpuscles, 20 μl Anti-r antibodies with 0.2 μg, protein/μl antibody solution, for example in PBS, which specifically bond to antigens of the surfaces of the nucleated stem cells of red blood corpuscles, and

20 μl Anti-i antibodies with 0.2 μg, protein/μl antibody solution, preferably in PBS, which specifically bond to the fetal Antigen-i on red stem cells.

The mixture ratio amounts to 20.0 μl, antibody cocktail to 10⁵-10⁹, preferably 10⁷ nucleated cells in the cell preparation.

In a further preparation of a practical example the antibody cocktail contains the following as antibodies for simultaneous incubation with the cell fraction:

20 μl Anti-w antibodies with 0.2 μg, protein/μl antibody solution, preferably in PBS, which specifically bond to antigens of the surface of white blood corpuscles,

20 μl Anti-r antibodies with 0.2 μg, protein/μl antibody solution, preferably in PBS, which specifically bond to antigens of the surfaces of the nucleated stem cells of red blood corpuscles, and

20 μl Anti-i antibodies with 0.2 μg, protein/μl antibody solution, preferably in PBS, which specifically bond to the fetal Antigen-i on red stem cells, and/or 20 μl Anti-i plus antibodies with 0.2 μg, protein/μl antibody solution, preferably in PBS, toward, membrane fragments of fetal red stem cells, preferably also containing Antigen-i on the membrane surface, which are specific for the fetal nucleated cells. The mixture ratio amounts to 20.0 μl, antibody-cocktail to 10⁷ nucleated cells in the cell preparation.

In a further preparation of a practical example the antibody cocktail contains only the Anti-w and Anti-r antibodies, which are first incubated with the cell fraction, whereby then the additions of the Anti-i and/or the Anti-i-plus antibodies occur in succession to the antibody cocktail cell fraction:

Antibody-Cocktail.

20 μl Anti-w antibodies with 0.2 μg, protein/μl antibody solution, preferably in PBS, which specifically bond to antigens of the surface of white blood corpuscles, 20 μl Anti-r antibodies with 0.2 μg, protein/μl antibody solution, preferably in PBS, which specifically bond to antigens of the surfaces of the nucleated stem cells of red blood corpuscles, whereby the mixture ratio amounts to 20.0 μl, antibody-cocktail to 10⁵-10⁹, preferably 10⁷ nucleated cells in the cell preparation.

Then the antibody cocktail cell fraction has the following added to it: either 20 μl Anti-i antibodies with 0.2 μg, protein/μl antibody solution, preferably in PBS, which specifically bond to the fetal Antigen-i on red stem cells, or 20 μl Anti-i plus antibodies with 0.2 μg, protein/μl antibody solution, preferably in PBS, toward, membrane fragments of fetal stem cells, preferably also containing Antigen-i on the membrane surface, which are specific for the fetal nucleated cells.

In a further practical example a mixture of the following is added to the antibody cocktail cell fraction

20 μl Anti-i antibodies with 0.2 μg, protein/μl antibody solution, preferably in PBS, which specifically bond to the fetal Antigen-i of red stem cells, and 20 μl Anti-i plus antibodies with 0.2 μg, protein/μl antibody solution, preferably in PBS, toward, membrane fragments of fetal stem cells, preferably also containing Antigen-i on the membrane surface, which are specific for the fetal nucleated cells.

An antibody with a specific bond to Lacto-N-nor-hexaosyl-Ceramide and an Anti-i antibody, which is provided from B-Lymphocytes of a vertebrate immunized against Lacto-N-nor-hexaosylceramide of the native type and against membrane fragments of fetal cells, such as a mouse and/or rabbit , and a further mouse and/or rabbit provided from B-Lymphocytes immunized against Lacto-N-nor-hexaosylceramide of the synthetic type are provided as Anti-i antibodies.

An antibody with a specific bond to membrane fragments, intracellular structures and/or intracellular molecules of fetal red stem cells and an Anti-i-plus antibody which stems from B-Lymphocytes of a mouse and/or other species of e.g. vertebrates immunized against membrane fragments, intracellular structures and/or intracellular molecules of fetal red stem cells are provided as Anti-i-plus antibodies.

In a further practical example the whitish buffy-coat-layer as cell fraction (e.g. 1-4×10⁷ cells) is incubated with an antibody cocktail made of antibodies of the polyclonal type at room temperature for 20 min long to isolate the cell preparation. The antibody cocktail with polyclonal antibodies enriched or isolated from rabbits, sheep or horses contains fluorescein, phycoerythrine and/or peridinin chlorophyll protein (PerCP) marked antibodies (20 μl per antibody) of the polyclonal type with 0.2 μg protein/μl antibody cocktail in PBS with the above named compositions and the incubation conditions occur as above with those with monoclonal antibodies.

After the incubation with polyclonal and/or monoclonal antibodies, the same are separated from the cell preparation with the help of the flow cytometer (e.g. Becton Dickinson type, Vantage SE) using the transmitted light, diffuse light, and fluorescent properties of the fetal cells bound by means of fluorescent marked antibodies as well as also by their cell size and their available cell compartments with up to 10⁵ to 10⁷ isolation of the same.

1. Procedure for enrichment of fetal cells as a product of peripheral maternal blood, whereby in
Step a) after a withdrawal of maternal blood in the presence of a solution containing one or more anticoagulants for preparation of a blood fraction, the blood is diluted with an isotonic solution as well as in Step b) the blood fraction, for the purpose of enrichment of a cell fraction with nucleated fetal cells, is centrifuged, characterized by the fact that in
Step c) an antibody cocktail made of antibodies of the polyclonal and/or monoclonal type, preferably at RT for 15 to 30 min, even more preferably 10 to 20 min long which contains Anti-w antibodies, which specifically bond to antigens of the intracellular structures of white blood corpuscles and/or the intracellular molecules of white blood corpuscles and/or surfaces of white blood corpuscles, and
Anti-r antibodies, which specifically bond to antigens of the intercellular structures of nucleated stem cells of red blood corpuscles and/or the intracellular molecules of nucleated stem cells of red blood corpuscles and/or the surfaces of nucleated stem cells of red blood corpuscles, is added to the cell fraction and then
Anti-i antibodies of the polyclonal and/or monoclonal type, which specifically bond to Antigen-i as antigens of the surfaces of nucleated stem cells of red blood corpuscles, and/or
Anti i plus antibodies of the polyclonal and/or monoclonal type, which specifically bond to intracellular structures of fetal stem cells and/or the intracellular molecules of fetal stem cells and/or membrane fragments of fetal stem cells, or adds mixtures of the same, under formation of a cell preparation, which contains antibody-complexes, as well as
Step d) fetal cells with Anti-r antibodies and Anti-i antibodies coupled to the same, and/or Anti-i plus antibodies, on the basis of their diffuse light and fluorescent properties, by the properties of surfaces and of cell interiors as well as the size distribution of the fetal cells with 10⁵ to 10⁷ enrichment of the fetal cells, are separated from the cell-preparation.

2. Procedure for the enrichment of fetal cells as the product of peripheral maternal blood as set forth in claim 1; characterized by the fact that in Step c) the antibody cocktail made of antibodies of the polyclonal and/or monoclonal type is added to the cell fraction at RT for 5 to 30 min long.

3. Procedure for the enrichment of fetal cells as the product of peripheral maternal blood as set forth in claim 1 or 2; characterized by the fact that in Step c) the antibody cocktail made of antibodies of the polyclonal and/or monoclonal type is added to the cell fraction at RT for 10 to 20 min long.

4. Procedure for the enrichment of fetal cells as the product of peripheral maternal blood as set forth in one of claims 1 to 3; characterized by the fact that in Step d) fetal cells with Anti-r antibodies and Anti-i antibodies coupled to the same, and/or Anti-i plus antibodies, on the basis of their diffuse light and fluorescent properties, by the properties of surfaces and of cell interiors as well as the size distribution of the fetal cells with 10⁵ to 10⁷ enrichment of the fetal cells, are separated from the cell-preparation.

5. Procedure for the enrichment of fetal cells as the product of peripheral maternal blood as set forth in one of claims 1 to 5; characterized by the fact that in Step a) venous and/arterial maternal blood is withdrawn.

6. Procedure for the enrichment of fetal cells as the product of peripheral maternal blood as set forth in one of claims 1 to 5; characterized by the fact that in Step a) the blood is diluted with a solution containing NaCl with a buffer of pH 7.2 to 7.4.

7. Procedure for the enrichment of fetal cells as the product of peripheral maternal blood as set forth in one of claims 1 to 6; characterized by the fact that in Step b) the blood fraction is centrifuged at 800×g for 0.1 to 60 min long.

8. Procedure for the enrichment of fetal cells as the product of peripheral maternal blood as set forth in claim 7; characterized by the fact that in Step b) the blood fraction is centrifuged at 800×g for 15 to 60 sec long.

9. Procedure for the enrichment of fetal cells as the product of peripheral maternal blood as set forth in one of claims 1 to 8; characterized by the fact that as antibodies, polyclonal antibodies from vertebrates, preferably horses, sheep and/or rabbits, are used.

10. Procedure for the enrichment of fetal cells as the product of peripheral maternal blood as set forth in one of claims 1 to 9; characterized by the fact that as antibodies, monoclonal antibodies from vertebrates, preferably mice and/or rats, are used.

11. Procedure for the enrichment of fetal cells as the product of peripheral maternal blood as set forth in one of claims 1 to 10; characterized by the fact that in Step d) with the help of a flow cytometer, fetal cells with Anti-r and Anti-i antibodies bound to the cell surface are separated from the cell preparation.

12. Procedure for the enrichment of fetal cells as the product of peripheral maternal blood as set forth in one of claims 1 to 11; characterized by the fact that in Step d) with the help of a flow cytometer, fetal cells with Anti-r,
Anti-i and/or Anti-i-plus antibodies bound to the cell surface are separated from the cell preparation.

13. Procedure for the enrichment of fetal cells as the product of peripheral maternal blood as set forth in one of claims 1 to 12; characterized by the fact that an antibody with a specific bond to Lacto-N-nor-hexaosylceramide is used as the Anti-i antibody.

14. Procedure for the enrichment of fetal cells as the product of peripheral maternal blood as set forth in one of claims 1 to 13; characterized by the fact that a monoclonal antibody which is provided from B-Lymphocytes of a vertebrate immunized against Lacto-N-nor-hexaosylceramide of the native type, preferably a mouse, rat, and/or rabbit is used as the Anti-i antibody.

15. Procedure for the enrichment of fetal cells as the product of peripheral maternal blood as set forth in one of claims 1 to 14; characterized by the fact that a monoclonal antibody which is provided from B-Lymphocytes of a vertebrate immunized against Lacto-N-nor-hexaosylceramide of the chemically synthesized type, preferably a mouse and/or rat is used as the Anti-i antibody.

16. Procedure for the enrichment of fetal cells as the product of peripheral maternal blood as set forth in one of claims 1 to 15; characterized by the fact that a polyclonal antibody which is isolated from the blood of a vertebrate immunized against Lacto-N-nor-hexaosylceramide of the native type, preferably a rat, mouse and/or rabbit is used as the Anti-i antibody.

17. Procedure for the enrichment of fetal cells as the product of peripheral maternal blood as set forth in one of claims 1 to 16; characterized by the fact that a polyclonal antibody which is isolated from the blood of a vertebrate immunized against Lacto-N-nor-hexaosylceramide of the chemically synthesized type, type, preferably a mouse and/or rat is used as the Anti-i antibody.

18. Procedure for the enrichment of fetal cells as the product of peripheral maternal blood as set forth in one of claims 1 to 17; characterized by the fact that a monoclonal antibody which is provided from B-Lymphocytes of a vertebrate immunized against intracellular structures of fetal stem cells and/or intracellular molecules of fetal stem cells and/or membrane fragments of fetal stem cells, preferably rat, mouse and/or rabbit, is used as the Anti-i-plus antibody.

19. Procedure for the enrichment of fetal cells as the product of peripheral maternal blood as set forth in one of claims 1 to 16; characterized by the fact that a polyclonal antibody which is isolated from the blood of a vertebrate immunized against intracellular structures of fetal stem cells and/or intracellular molecules of fetal stem cells and/or membrane fragments of fetal stem cells, preferably mouse, rat and/or rabbit, is used as the Anti-i-plus antibody.

20. Application of the product as set forth in one of claims 1 to 19 for the treatment of heredity diseases.

21. Application of the product as set forth in claim 20 for the treatment of heredity diseases in the fetal period.

22. Application of the product as set forth in one of claims 1 to 19 for genome research.

23. Application of the product as set forth in claim 22 for genome research of fetuses.

24. Fetal cells of venous and/or arterial, maternal blood, producible, through in
Step a) dilution of blood gained after a withdrawal of maternal blood in the presence of an isotonic solution containing one or more anticoagulants for provision of a blood fraction as well as
Step b) centrifugation of the blood fraction, for the purpose of enrichment of a cell fraction with nucleated fetal cells,
Step c) Incubation of the cell fraction with an antibody cocktail with antibodies of the polyclonal and/or monoclonal type, at RT for 15 to 30 min, preferably 10 to 20 min long which contains Anti-w antibodies, which specifically bond to antigens of the intracellular structures of white blood corpuscles and/or the intracellular molecules of white blood corpuscles and/or surfaces of white blood corpuscles, and
Anti-r antibodies, which specifically bond to antigens of the intercellular structures of nucleated stem cells of red blood corpuscles and/or the intracellular molecules of nucleated stem cells of red blood corpuscles and/or the surfaces of nucleated stem cells of red blood corpuscles, and with
Anti-i antibodies of the polyclonal and/or monoclonal type, which specifically bond to Antigen-i as antigens of the surfaces of nucleated stem cells of red blood corpuscles, and/or
Anti i plus antibodies of the polyclonal and/or monoclonal type, which specifically bond to intracellular structures of fetal stem cells and/or the intracellular molecules of fetal stem cells and/or membrane fragments of fetal stem cells, under formation of a cell preparation made of antibody-complexes that comprise antibodies Anti-r, Anti-i and/or Anti-i-plus antibodies coupled to fetal cells and
Step d)separation of the fetal cells with the antibodies coupled to the same from the cell-preparation on the basis of their diffuse light and fluorescent properties, according to the properties of surfaces and of cell interiors as well as the size distribution with 10⁵ to 10⁷ enrichment of the fetal cells.

25. Fetal cells of venous and/or arterial, maternal blood as set forth in claim 24, characterized by the fact that the additions of the antibody cocktail, the Anti-i and/or the Anti i plus antibody occurs either simultaneously or in succession.

26. Fetal cells of venous and/or arterial, maternal blood as set forth in claim 24 or 25, producible through in Step c) incubation of the cell fraction with the antibody cocktail of antibodies of the polyclonal and/or monoclonal type at RT for 10 to 20 min long.

27. Fetal cells of venous and/or arterial, maternal blood as set forth in one of claims 24 to 26, whereby a monoclonal antibody which is produced from B-Lymphocytes of a vertebrate immunized against Lacto-N-nor-hexaosylceramide of the native type and/or derivatives of the native type, preferably a mouse, and/or rat and/or
which is produced from B-Lymphocytes of a vertebrate immunized against Lacto-N-nor-hexaosylceramide of the chemically synthesized type and/or derivatives of the chemically synthesized type, preferably a mouse, and/or rat is used as the Anti-i antibody.
